(19) United States
(12) Patent Application Publication
(10) Pub. No.: US 2019/0134388 A1
(43) Pub. Date: May 9, 2019

(54) TENS DEVICE

(71) Applicant: HOLLYWOG, Chattanooga, TN (US)

(72) Inventors: Charles M. Thomas, Hixson, TN (US); D. Keith Hagy, Hixson, TN (US); Michael W. Treas, Chattanooga, TN (US); Jeffrey J. Chu, Norwich, VT (US); Mike Ewaschuk, Hartford, VT (US); Josh Page, (US); Dave Eypper, Fairlee, VT (US)

(21) Appl. No.: 15/808,724

(22) Filed: Nov. 9, 2017

(51) Int. Cl. A61N 1/36; A61N 1/04

(52) U.S. Cl. CPC A61N 1/3603; A61N 1/36021; A61N 1/0456

(57) ABSTRACT

A transcutaneous electrical nerve stimulation device includes a housing having a bottom portion and a top portion, the top portion including a curved palm-rest surface shaped to conform generally to the shape of a human palm of a human hand when the human hand is partially opened, a first switch arranged on the top portion of the housing, the first switch being positioned to align in facing contact with a first finger of the human hand when the human hand is grasping the device, the first switch being operable by generally downward vertical movement of the first finger of the human hand, and an electrode operative to emit a signal responsive to an activation of the first switch, the electrode arranged on the bottom portion of the housing.

### BACKGROUND

The present invention generally relates to transcutaneous electrical nerve stimulation (TENS) devices, and more specifically, to hand-held TENS devices.

Transcutaneous electrical nerve stimulation is a therapeutic procedure that uses electrical current to stimulate nerves that provides a therapeutic benefit to patents. The therapeutic benefits of TENS devices are often used to provide therapy that may suppress or prevent pain in patients.

Previous TENS devices include adhesive pads that a user adheres to the skin to secure electrodes. The electrodes are connected to a power source and controller that modulates the pulse width, amplitude, and frequency of signals. The signals pass between the electrodes via the skin or other tissue thereby stimulating nerves and providing therapeutic benefits to the patent.

### SUMMARY

According to one embodiment, a transcutaneous electrical nerve stimulation device includes a housing having a bottom portion and a top portion, the top portion including a curved palm-rest surface shaped to conform generally to the shape of a human palm of a human hand when the human hand is partially opened, a first switch arranged on the top portion of the housing, the first switch being positioned to align in facing contact with a first finger of the human hand when the human hand is grasping the device, the first switch being operable by generally downward vertical movement of the first finger of the human hand, and an electrode operative to emit a signal responsive to an activation of the first switch, the electrode arranged on the bottom portion of the housing.

According to another embodiment, a transcutaneous electrical nerve stimulation device includes a housing having a bottom portion and a top portion, the top portion including a curved palm-rest surface shaped to conform generally to the shape of a human palm of a human hand when the human hand is partially opened, a first switch arranged on the top portion of the housing, the first switch being positioned to align in facing contact with a first finger of the human hand when the human hand is grasping the device, the first switch being operable by generally downward vertical movement of the first finger of the human hand, and an electronic portion partially arranged in the housing, the electronic portion includes a power source, a boost converter communicatively connected to the power source, a timer communicatively connected to the boost converter and a switching device, a transformer communicatively connected to the boost converter and the timer, and an electrode operative to emit a signal responsive to an activation of the first switch, the electrode arranged on the bottom portion of the housing.

According to yet another embodiment, a transcutaneous electrical nerve stimulation device includes a housing having a bottom portion and a top portion, the top portion including a curved palm-rest surface shaped to conform generally to the shape of a human palm of a human hand when the human hand is partially opened, a first switch arranged on the top portion of the housing, the first switch being positioned to align in facing contact with an index finger of the human hand when the human hand is grasping the device, the first switch being operable by generally downward vertical movement of the index finger of the human hand, and an electrode operative to emit a signal responsive to an activation of the first switch, the electrode arranged on the bottom portion of the housing.

### BRIEF DESCRIPTION OF THE DRAWINGS

FIG. 1 illustrates a perspective view of an exemplary embodiment of a TENS device.

FIG. 2 illustrates another perspective view of the device.

FIG. 3 illustrates a top view of the device.

FIG. 4 illustrates a bottom view of the device.

FIG. 5 illustrates a side-partially exploded view of the device.

FIG. 6 illustrates a side view of a manner of using the device

FIG. 7 illustrates a user operating the device.

FIG. 8 illustrates a side view of the electronic portion that is at least partially disposed in a cavity defined by the housing.

FIG. 9 illustrates a side-partially cut-away view of the device along the line A-A of FIG. 3).

FIG. 10 illustrates a block diagram of an exemplary embodiment of the electronic portion (of FIG. 5) of the device (of FIG. 1).

FIG. 11 illustrates a bottom view of an alternate exemplary embodiment of a device.

FIG. 12 illustrates an example of a wave form of a first signal output by the device (of FIG. 1).

FIG. 13 illustrates an example of a wave form of a second signal output by the device (of FIG. 1).

FIG. 14 illustrates an example of a wave form of a third signal output by the device (of FIG. 1).

### DETAILED DESCRIPTION

The embodiments described herein provide for a hand-held transcutaneous electrical nerve stimulation (TENS) device that provides an ergonomic, simple, and effective user interface in a relatively inexpensive device.

As discussed above, previous TENS devices often use a number of adhesive pads that are secured to electrodes that are arranged in contact with or proximate to the skin. The adhesive pads are adhered to the skin, and are connected with wires to the TENS devices. The use of adhesive pads may be expensive, uncomfortable, and inconvenient for many users. The use of many TENS devices may be impractical when a user is away from home or in public due to the nature of applying the adhesive pads to the skin and connecting the adhesive pads to a bulky control unit with wires, which may be challenging when a user is wearing clothing.

Further, previous TENS devices are generally expensive for casual users to purchase and maintain. Such devices often include complicated user controls that are difficult for untrained users to operate.

The therapeutic effects of TENS devices are dependent on a number of factors. For example, the part of the body where the electrodes are placed affects the therapeutic effect. The size and relative location of the electrodes affects the therapy, and the properties of the waveform (e.g., amplitude, frequency, and pulse width) of the TENS device also affect the therapeutic effect felt by users. Varying pressure exerted on the skin with the electrodes may contribute to the effectiveness of the TENS therapy.

The embodiments described herein provide for a hand-held TENS device that is designed to be easily used, maintained, and carried by a user. In this regard, the embodiments described herein provide for a device having electrodes that may be placed in contact with the skin or clothing proximate to the skin without using adhesive pads.

As discussed above, the location of the electrodes, and the pressure applied by the user when using the device to place the electrodes in contact with (or arranged proximate to the skin with, for example, a layer(s) of clothing or another object disposed between the contacts and the electrodes) the skin may affect the therapy received by the user.

Thus, it is desirable for a hand-held TENS device that is ergonomically designed to allow a user to easily position the electrodes on or in proximity to the skin using the fingertip (e.g., index finger) of the user, which provides a natural and intuitive alignment and actuation method. Such an ergonomic arrangement allows a user to induce a force through the actuation switch of the device to the electrodes arranged below the actuation switch, thereby providing the user with the ability to adjust the force applied to the skin while actuating the device with the finger of the user. Such an embodiment also provides greater reach or range of motion, as opposed to other less ergonomic device arrangements, for the user to position the electrodes, actuate the actuation switch of the device, and exert a desired mechanical force on the device. The mechanical force exerted is exerted by the user using the index finger of the user to provide a desired amount of pressure on the body (e.g., skin), which may improve the therapeutic effect of the device while operating.

The embodiments described herein provide for an ergonomic arrangement of the actuation button and the electrodes such that a user may hold the device in the hand of the user, placing the index finger of the user over the actuation button, which is arranged such that the electrodes are substantially in-line with the actuation button. The user may place the electrodes of the device in contact with or proximate to the skin (e.g., with clothing disposed between the skin and the electrodes) and actuate the actuation button with their index finger by increasing a force applied by the index finger to the actuation button. Such force is applied along a vector that passes through the actuation button, electrodes, and the skin of the user such that the pressure on the actuation button also induces force or pressure on the skin via the electrodes.

Though using the index finger may offer increased “reach” by a user, other fingers of either hand including the thumb may be used to operate and actuate the device 100.

The embodiments further provide a TENS device having an easily adjusted intensity or voltage setting for a user such that a user may easily change the intensity of the wave forms emitting during a therapy session.

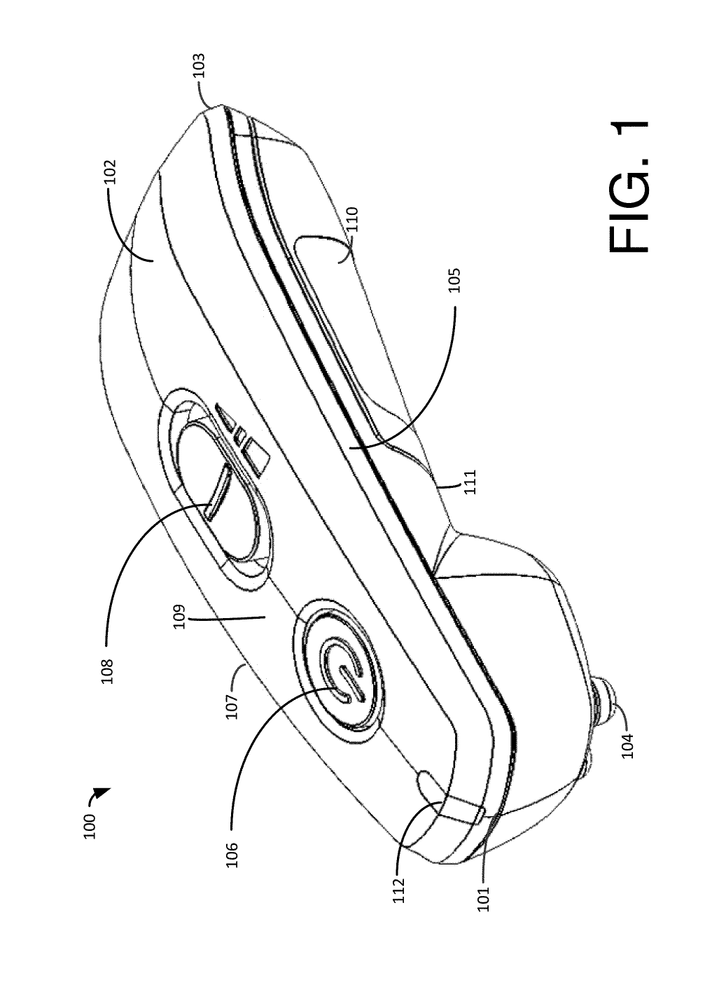

In this regard, FIG. 1 illustrates a perspective view of an exemplary embodiment of a TENS device (device) 100. The device 100 includes a housing 102, electrodes 104, an actuation button 106, an optional mode selection button 108, and an electronic portion (described below) arranged in an interior cavity of the housing 102.

The housing 102 includes a first distal end 101, a second distal end 103, a first lateral side 105, a second lateral side 107, a top portion 109, and a bottom portion 111. A battery access cover 110 may be attached to the housing 102 to retain and cover a battery (described below). The illustrated exemplary embodiment includes a visual indicator 112 such as, for example, a light emitting diode that emits a visual indicator corresponding to the modes or actuation of the device 100.

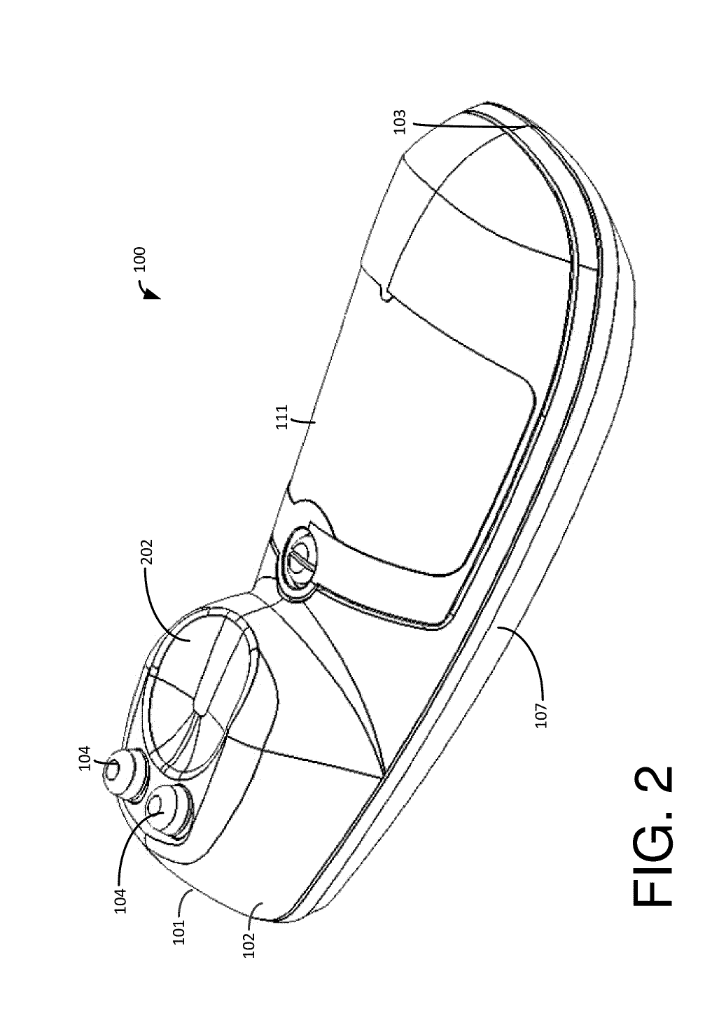

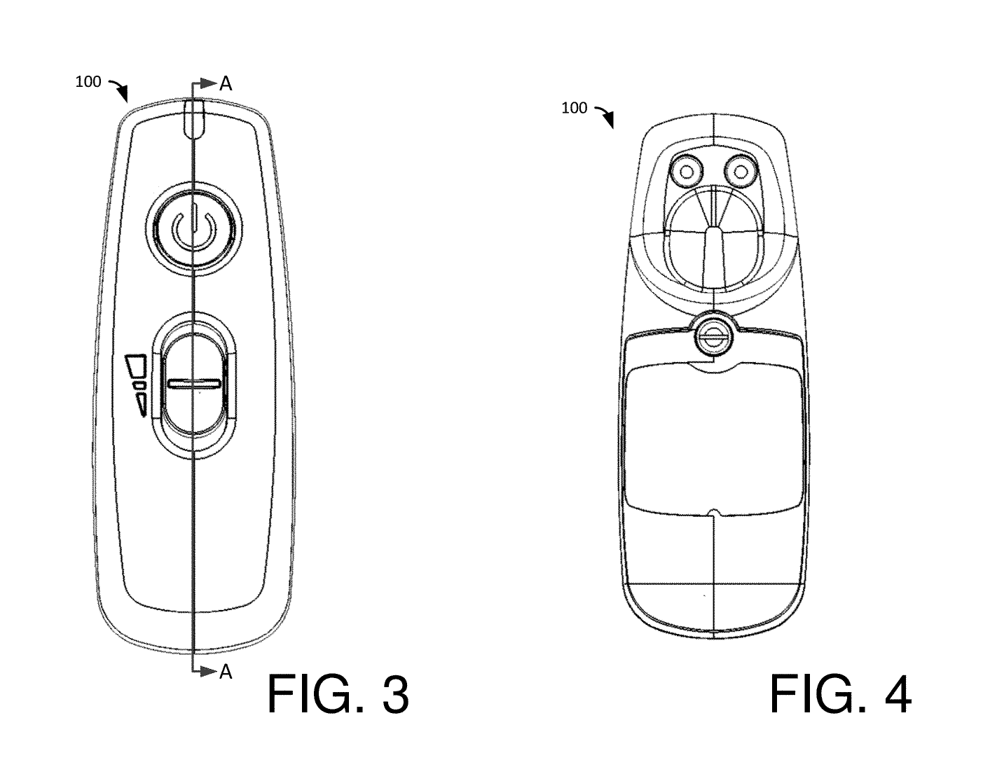

FIG. 2 illustrates another perspective view of the device 100, FIG. 3 illustrates a top view of the device 100, and FIG. 4 illustrates a bottom view of the device 100. FIG. 2 shows a recess (cavity) 202 that is arranged proximate to the electrodes 104. The recess 202 provides clearance or space for parts of a user to fit such that the electrodes 104 contact a desired portion of the user. For example, the user may place the electrodes 104 on a location on a hand of the user, a knuckle of the hand may be arranged in the recess 202 to facilitate the user placing the electrodes 104 in the desired location.

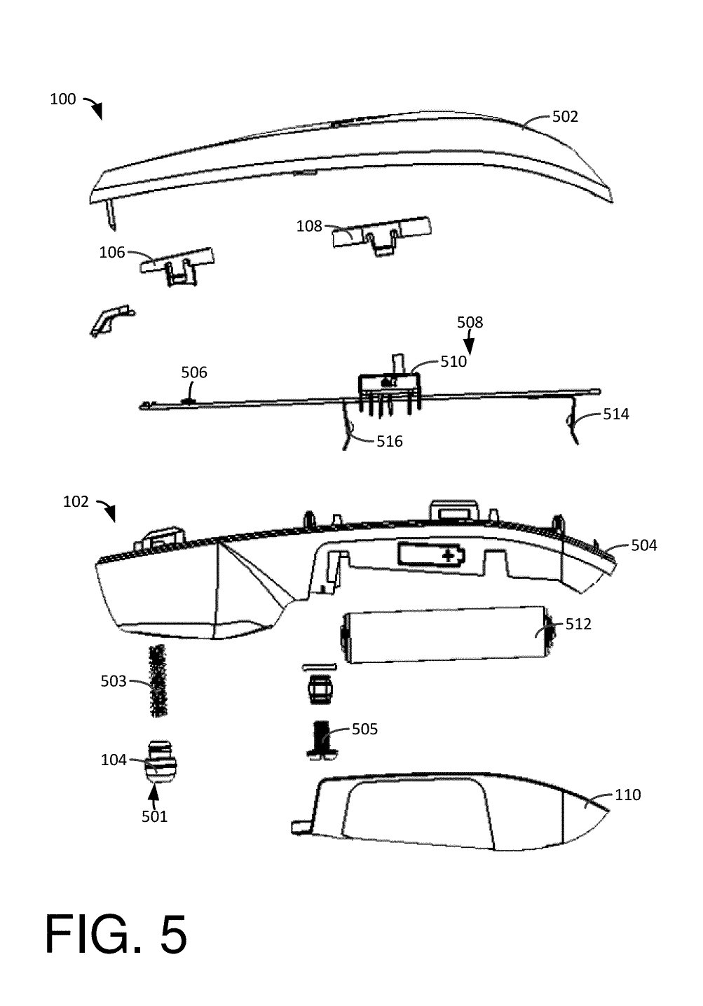

FIG. 5 illustrates a side-partially exploded view of the device 100. In this regard, the illustrated exemplary embodiment includes the housing portion 102 that includes an upper portion 502 and a lower portion 504. The battery access cover 110 is operative to be slidably or compressively secured and retained by the lower portion 504. Some exemplary embodiments may include fasteners 505 that secure the battery access cover 110. The lower portion 504 and the upper portion 502 may be assembled using any suitable method such as, for example, adhesives, plastic welding methods, fasteners, a snap-fit, or a press-fit securing arrangement. The actuation button 106 is arranged to engage an actuation switch 506 arranged on the electronic portion 508. In the illustrated exemplary embodiment, the actuation button 106 is arranged to slidably or hingably engage the actuation switch 506. The mode selection button 108 is arranged to engage a mode selection switch 510 arranged on the electronic portion 508. The electrode 104 having an outer facing surface 501 is connected to the electronic portion 508. The electrode 104 is connected to a biasing member 503 that may include, for example, a spring or another suitable type of biasing member or wire member. A battery(s) 512 is operative to engage battery terminals 514 and 516. The housing portion 102 may be formed from, for example, a plastic or polymer material or another suitable material.

In some exemplary embodiments, the electrodes 104 may be secured to the lower portion 504 such that the electrodes 104 do not move relative to the lower portion 504. The biasing member 503 is operative to provide an electrical connection between the electrodes and the electronic portion 508.

In an alternate exemplary embodiment, the biasing members 503 are operative to bias the electrodes 104 independently of each other such that the electrodes 104 are operative to move relative to the lower portion 504. Such a feature provides for improved contact between the electrodes and an uneven or non-planar surface. In such an embodiment, the biasing member 503 may be operative to provide an electrical connection between the electrode 104 and the electronic portion 508.

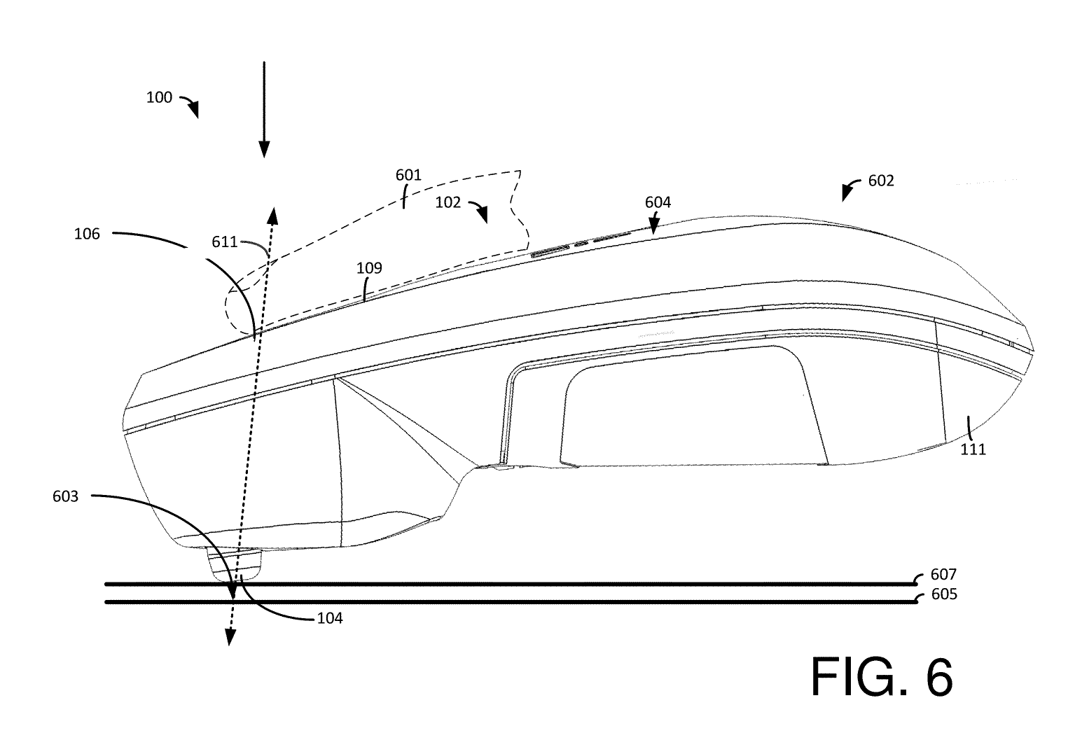

FIG. 6 illustrates a side view of a manner of using the device 100. A manner of using the device 100 is to grasp a grip portion 602 of the housing portion 102 in the human hand with the index finger 601 positioned on the actuation button 106. The middle, ring, and little fingers wrap around the bottom portion 111 while the palm of the hand rests on a palm-rest surface 604 of the top portion 109 of the grip portion 602.

In use, the user places the device such that the electrodes 104 are in contact with (over/above) or proximate to a target area 603 of the skin 605 (a layer(s) of clothing 607 may be disposed between the electrodes 104 and the skin). The target area 603 is a region of the skin on the body of a user that will receive the TENS therapy.

As discussed above, the pressure or force exerted by the user when depressing or actuating the actuation button 106 provides for user control of the pressure or force applied to the skin 605 such that greater force applied by the user to the actuation button 106 increases the pressure applied by the electrodes 104 on the skin 605. An example of the force is shown in FIG. 6 by a vector 611 that passes through the actuation button 106 and the electrodes 104. Such pressure may place the electrodes in closer proximity to the skin 605 by deforming the clothing 607. Such pressure may also deform the outer surface of the skin 605 due to the elastic nature of the skin 605. Often users experience a greater intensity during the therapy session when greater pressure is applied to the skin 605. Thus, the therapeutic benefits or effects of the device 100 may be partially related to the pressure or force exerted by the user when depressing the actuation button 106.

The position of the electrodes proximate to the target area 603 and the ability of the user to both operate the device 100 by, for example, actuating the actuation button 106 and/or the mode selection button 108, and applying a desired amount of pressure or force to the device 100 through the actuation button 106 is partially dependent on the ergonomic arrangement of the device. That the device 100 is arranged such that the hand of the user may grip the grip portion 602 with the index finger extended in a comfortable and natural position (i.e., a pointing position) allows a user to access a greater range of target areas 603 on the body of the user, greatly improving the usefulness of the device 100 by providing TENS therapy to areas of the body where the user may easily and naturally touch with their index finger. Such a feature improves the operation and usefulness of the device as opposed to an arrangement that uses another digit such as the thumb to actuate the actuation button 106 by increasing the areas of the skin that are reachable for applying self-therapy by the user.

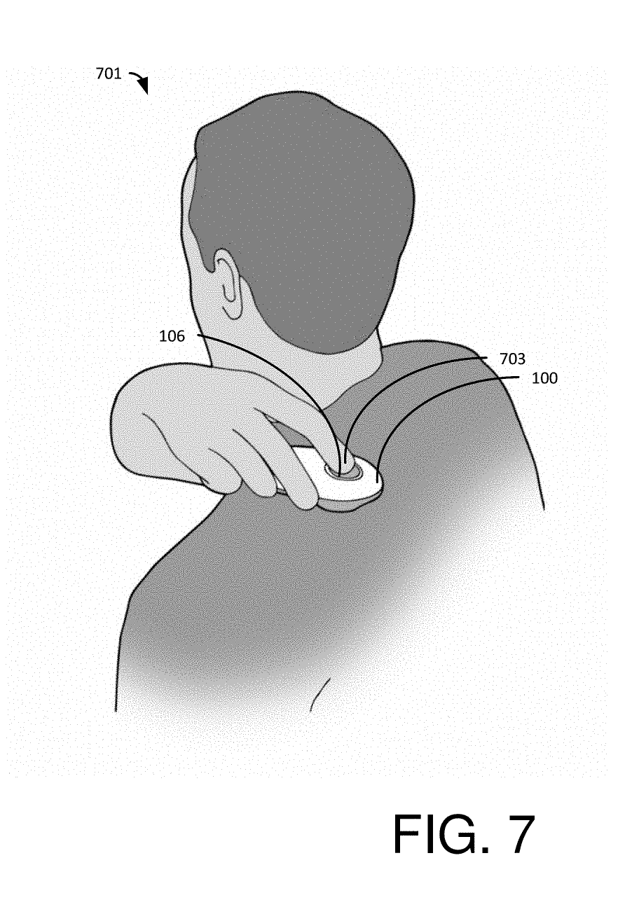

FIG. 7 illustrates a user 701 operating the device 100 in a similar manner as described above. In this regard, the user 701 has positioned the device 100 on a target area of the back of the user 701 that would be challenging for the user 701 to reach using a finger other than the index finger 703 to actuate the actuation button 106. In other words, the ergonomic relative geometric relationship between the grip portion 602 (of FIG. 6), the actuation button 106, and the electrodes 104 (of FIG. 6) provides a useful and real functional benefit to the user and improves the therapeutic benefits to the user by increasing the areas on the body that a user may reach while gripping the device as described above.

Some users may have health conditions or disabilities that limit their range of motion or use of one or both hands or arms. Thus, some users may only be able to use a single hand to operate the device 100. The limited use of a single hand typically restricts the areas of the body where a user may place a TENS device due to limited mobility therefore, ergonomically arranging the grip portion 602 (of FIG. 6), the actuation button 106, and the electrodes 104 (of FIG. 6) provides a greater range of motion and a greater “reach” for an impaired user. Such increased “reach” allows the device 100 to be used effectively by a greater number of users who may have been limited from using previous TENS devices due to their health conditions or other disabilities. The device 100 may be used ambidextrously with either hand of a user.

As discussed above, a user may also desire to vary the pressure exerted by the index finger of the user to vary the therapeutic effects of the device 100. Using other ergonomic arrangements generally limits both the reach of the user (i.e., the areas of the body accessible or reachable such that the user may position the electrodes over the target area while holding the device 100 as described above) and the ability of the user to apply a desired amount of pressure to the skin to desirably affect the therapeutic effects of the device 100. Independently, the position of the electrodes 104 of the device 100 relative to the target area 603 (of FIG. 6) and the pressure exerted on the skin 605 each have an appreciable effect on therapeutic effects of the device 100 when being operated by the user. Therefore, the embodiments of the device 100 described herein provide for an ergonomic grip arrangement that allows a user to both position the electrodes 104 of the device 100 over a challenging to reach target area 603 while simultaneously, or substantially simultaneously actuating the actuation button 106 with the index finger and applying a desired pressure with the index finger.

The extended shape of the grip portion 602 further provides a lever arm or pivoting arm that allows a user to further control or exert forces on the device using a natural and comfortable grip position as described above.

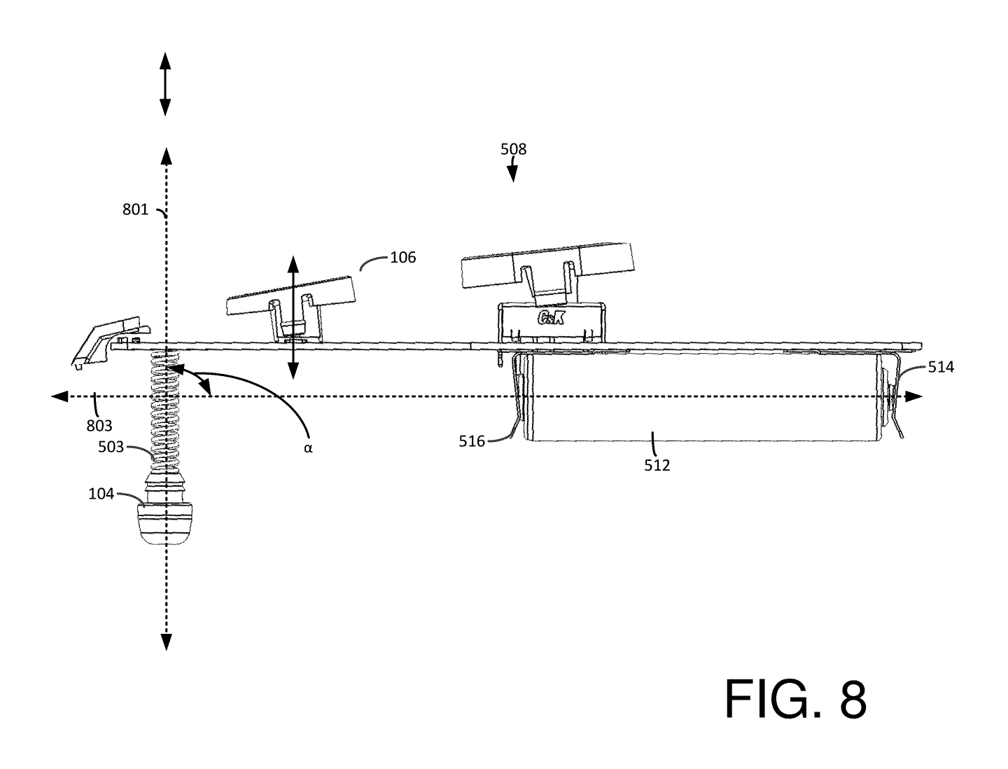

FIG. 8 illustrates a side view of the electronic portion 508 that is at least partially disposed in a cavity defined by the housing 102 (of FIG. 1). The electrodes 104 and biasing members 503 are arranged such that a plane depicted by the line 801 passes through the biasing members 503 and the electrodes 104. The activation switch 106 has a linear range of motion that is substantially parallel to the plane depicted by the line 801 such that a component of the force vector exerted on the activation button (switch) 106 is substantially parallel to the plane depicted by the line 801. The electronic portion 508 includes battery terminals 514 and 516 that are operative to contact terminals of a battery 512. The battery terminals 514 and 516 are arranged such that the line 803 passes through the battery terminals 514 and 516 and the battery 512. The battery 512 has a longitudinal axis that is substantially collinear with the line 803. The planes depicted by the lines 801 and 803 are shown intersecting and forming an angle (α). In the illustrated exemplary embodiment the angle α is approximately 90 degrees. In other exemplary embodiments, the angle α is less than 90 degrees, but greater than 0 degrees.

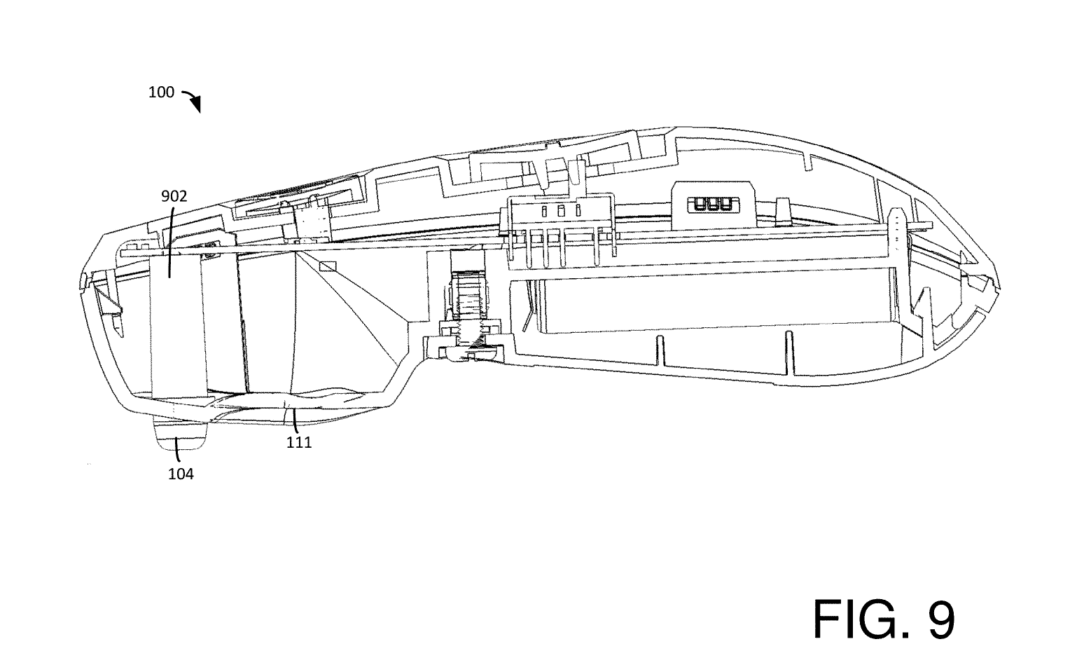

FIG. 9 illustrates a side-partially cut-away view of the device 100 along the line A-A of FIG. 3). The electrodes 104 of the device 100 are formed from a conductive material such, as for example, a metallic material. Each of the electrodes 104 is communicably connected to the electronic portion 508. The biasing members 503 are arranged in tubular cavities 902 defined in the bottom portion 111.

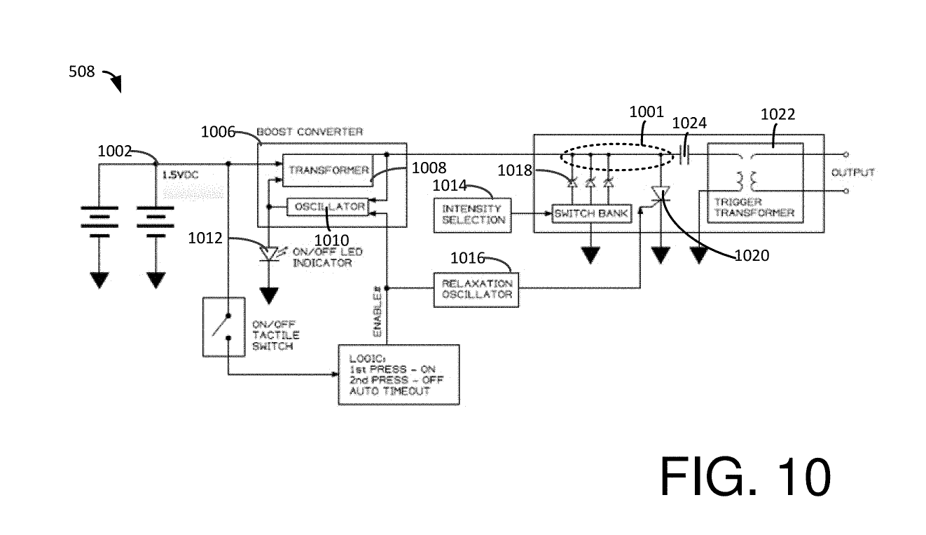

FIG. 10 illustrates a block diagram of an exemplary embodiment of the electronic portion 508 (of FIG. 5) of the device 100 (of FIG. 1). In this regard, the electronic portion 508 includes a power source portion 1002 that outputs power to a boost converter portion 1006. The power source portion of the illustrated exemplary embodiment includes a direct current (DC) power source such as, for example, a battery. Alternate exemplary embodiments may include, for example, a DC rectifying circuit that rectifies alternating current (AC) power to provide DC power. The illustrated exemplary embodiment may include, for example, a reverse polarity protection circuit portion or other power control, power conditioning, or other protection circuits.

In alternate exemplary embodiments, the electronic portion 508 of the device 100 may include a microprocessor or other type of logic device that is operative to control the device 100.

The boost converter portion 1006 includes a first transformer 1008 and an oscillator 1010 that are communicatively connected to a light emitting device (e.g., a light emitting diode (LED)) 1012, a user input and logic portion 1014, and a timer portion 1016. The output of the first transformer 1008 is communicatively connected to a voltage control portion 1018 and a switching portion 1020 at the node 1001.

An input of a second transformer 1022 is communicatively connected to the node 1001, and the output of the second transformer 1022 is communicatively connected to the electrodes 104 (of FIG. 1).

In operation, when a user actuates the actuation switch 106 (of FIG. 1), the boost converter 1006 receives a first DC voltage from the power source portion 1002. The boost converter 1006 is operative to step up the first DC voltage to a second DC voltage. The second DC voltage may be controlled by the voltage control portion 1018 via the mode selection button 108 (of FIG. 1) in some exemplary embodiments. Other exemplary may omit the mode selection button (switch) 108.

In this regard, the user may select from a group of operational modes by changing the position of the mode selection button 108. The operational modes in the illustrated exemplary embodiment include a variety of intensity modes that each has a particular associated voltage for the signal output by the device 100. The voltage control portion 1018 may include, for example, an array of diodes (e.g., Zener diodes), resistors, and capacitors that have values selected to result in a desired output voltage for the device 100 that is associated with the mode selected by the user. A number of diodes and resistors are arranged in parallel with the second transformer and are selectable by the mode selection button 108. The voltage control portion 1018 is operative to allow the user to select a mode such that the intensity of the therapy (e.g., the amplitude of the voltage) meets the desires of the user. In the illustrated exemplary embodiment, the user may change the position of the mode selection button 108 during a therapy session while the actuation button 106 is being depressed.

In some alternate exemplary embodiments, the mode selection button 108 may be integrated into the actuation switch 106. For example, the actuation switch 106 may include a pressure or force sensitive device such as, for example a resistive, magnetic, capacitive, mechanical, or piezoelectric switching or touch sensitive device. In this regard, such an embodiment of the device may change operational modes or output wave forms as the user provides increased or decreased pressure to the actuation/mode selection button 108.

In some other alternate exemplary embodiments, the mode selection may be performed by an amount of pressure or force that is exerted on the electrodes 104. In this regard, the electrodes 104 may be biased by the biasing members 503 or may be substantially fixed relative to the housing 102. A pressure or force sensitive device such as, for example a resistive, magnetic, capacitive, mechanical, or piezoelectric device is arranged to sense a force or pressure applied to the electrodes 104. Such force may be used to change operational modes or output wave forms as the user provides increased or decreased pressure to the electrodes 104.

The signal output by the voltage control portion 1018 is switched by a switching portion 1020 may include, for example, a thyristor or another semiconducting switching device that is controlled by the timer 1016. The signal may pass through a capacitive element 1024 prior to reaching the second transformer 1022 that increases the voltage of the signal and outputs the signal via the electrodes 104 (of FIG. 1).

Though the illustrated exemplary embodiments include three mode (intensity) settings, alternate embodiments may include any number of operational modes having any number of different types of output waveforms.

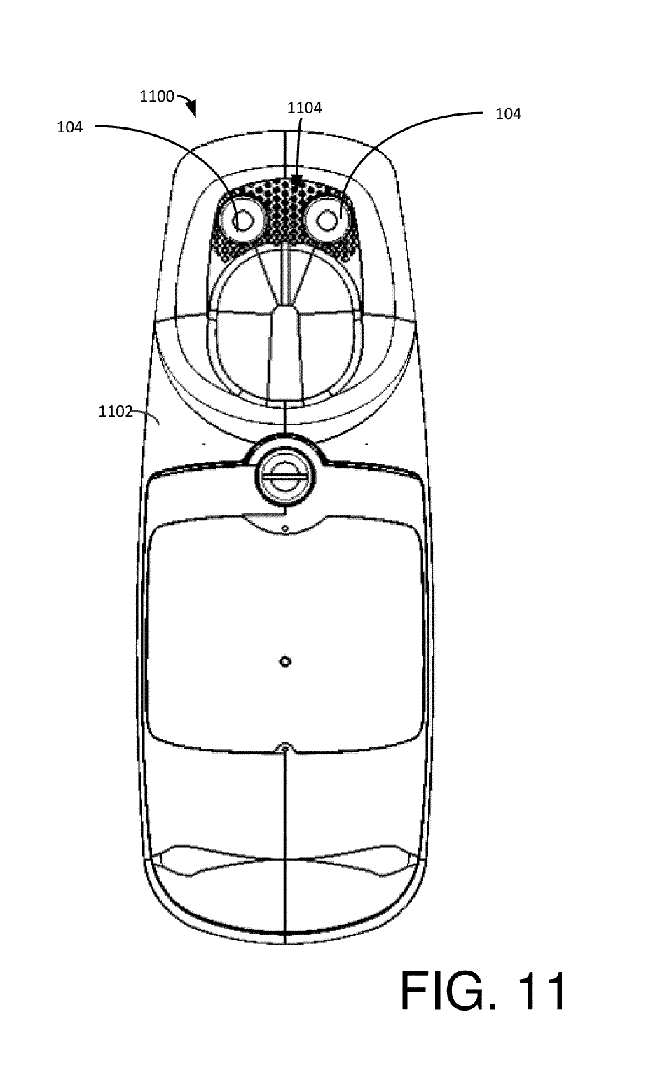

FIG. 11 illustrates a bottom view of an alternate exemplary embodiment of a device 1100. The device 1100 includes a housing 1102 having a textured surface region 1104 that is proximate to and between the electrodes 104. The textured surface region 1104 increases the surface area of the housing 1102 between and proximate to the electrodes 104. Such an arrangement is operative to increase the creep distance between the electrodes 104 to reduce, limit, or prevent, visible electrical arcing between the electrodes 104 when the device 1100 is in operation.

Though the illustrated exemplary embodiment of the device 1100 includes a textured surface region 1104 having dimples, indentations, or depressions, the textured surface region 1104 may include any type of textured design that is operative to increase the surface area of the housing 1102 in the region of the electrodes 104. For example, the textured surface region 1104 may include undulations, ribs, waves, fins, or any other type of feature that extends from or indents into the surface of the housing 1102.

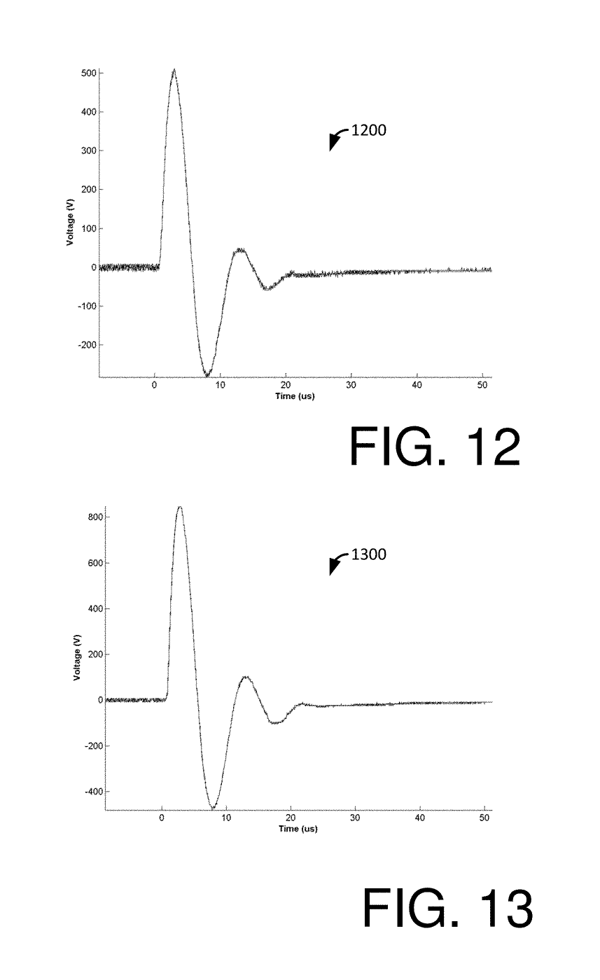

FIG. 12 illustrates an example of a wave form of a first signal output by the device 100 (of FIG. 1). In the illustrated exemplary embodiment, the first signal is associated with a first operational mode that may be selected by the user using the mode selection button 108 The first wave form 1200 of FIG. 11 shows an example of the first signal that is output by the device to provide therapy to a user. In this regard, the first signal measured in a circuit with a 2000 ohm load has a peak voltage of about 500 V and decays over approximately 25 milliseconds. The first signal has a net charge of approximately 1 uC and a net charge of about 0.17 uC to 0.39 uC.

FIG. 13 illustrates an example of a wave form of a second signal output by the device 100 (of FIG. 1). In the illustrated exemplary embodiment, the second signal is associated with a second operational mode that may be selected by the user using the mode selection button 108 The second wave form 1300 of FIG. 12 shows an example of the second signal that is output by the device to provide therapy to a user. In this regard, the second signal measured with a 2000 ohm has a peak voltage of about 850 V and decays over approximately 25 milliseconds. The second signal has a charge of between approximately 1 uC and 2 uC.

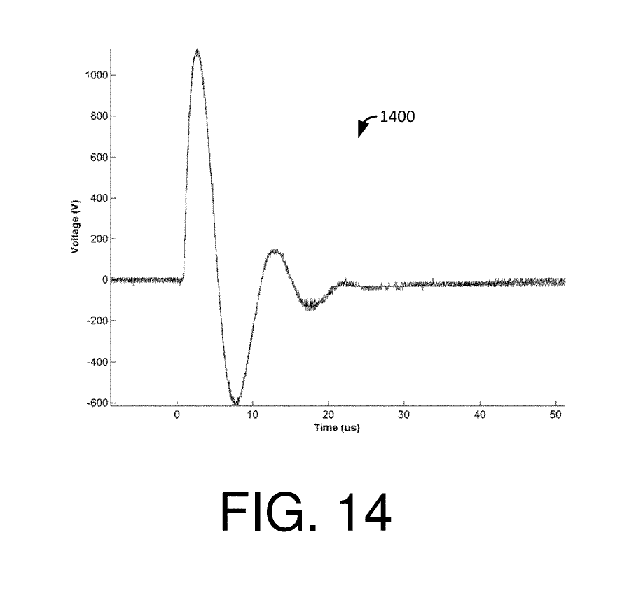

FIG. 14 illustrates an example of a wave form of a third signal output by the device 100 (of FIG. 1). In the illustrated exemplary embodiment, the third signal is associated with a third operational mode that may be selected by the user using the mode selection button 108 The third wave form 1400 of FIG. 14 shows an example of the third signal that is output by the device to provide therapy to a user. In this regard, the third signal measured with an open circuit has a peak voltage of about 1100 V and decays over approximately 25 milliseconds. The third signal has a charge of approximately 2 uC.

Though the signals shown in FIGS. 12-14 are emitted over a desired time period, alternate exemplary embodiments may operate in a mode that continuously outputs a signal while the actuation button 106 (of FIG. 1) is actuated by the user.

The embodiments described herein provide for a hand-held TENS device that is ergonomically designed to allow a user to easily position the electrodes on or in proximity to the skin using the fingertip (e.g., index finger) of the user, which provides a natural and intuitive alignment and actuation method while providing a desired amount of pressure using the fingertip of the user. Embodiments provide an economical, portable, and simple to operate TENS device having multiple modes of operation.

As used herein, the terms “invention” or “present invention” are non-limiting terms and not intended to refer to any single aspect of the particular invention but encompass all possible aspects as described in the specification and the claims. The term “on” can refer to an element that is on, above or in contact with another element or feature described in the specification and/or illustrated in the figures.

As used herein, the term “about” modifying the quantity of an ingredient, component, or reactant of the invention employed refers to variation in the numerical quantity that can occur, for example, through typical measuring and liquid handling procedures used for making concentrates or solutions. Furthermore, variation can occur from inadvertent error in measuring procedures, differences in the manufacture, source, or purity of the ingredients employed to make the compositions or carry out the methods, and the like. In one aspect, the term “about” means within 10% of the reported numerical value. In another aspect, the term “about” means within 5% of the reported numerical value. Yet, in another aspect, the term “about” means within 10, 9, 8, 7, 6, 5, 4, 3, 2, or 1% of the reported numerical value.

It will also be understood that when an element, such as a layer, region, or substrate is referred to as being “on” or “over” another element, it can be directly on the other element or intervening elements can also be present. In contrast, when an element is referred to as being “directly on” or “directly over” “on and in direct contact with” another element, there are no intervening elements present, and the element is in contact with another element.

It will also be understood that when an element is referred to as being “connected” or “coupled” to another element, it can be directly connected or coupled to the other element or intervening elements can be present. In contrast, when an element is referred to as being “directly connected” or “directly coupled” to another element, there are no intervening elements present.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A transcutaneous electrical nerve stimulation device comprising:
a housing having a bottom portion and a top portion, the top portion including a curved palm-rest surface shaped to conform generally to the shape of a human palm of a human hand when the human hand is partially opened;
a first switch arranged on the top portion of the housing, the first switch being positioned to align in facing contact with a first finger of the human hand when the human hand is grasping the device, the first switch being operable by generally downward vertical movement of the first finger of the human hand; and
an electrode operative to emit a signal responsive to an activation of the first switch, the electrode arranged on the bottom portion of the housing.

2. The device of claim 1, further comprising an electronic portion partially arranged in the housing, the electronic portion including:
a power source;
a boost converter communicatively connected to the power source;
a timer communicatively connected to the boost converter and a switching device;
a transformer communicatively connected to the boost converter and the timer; and
an electrode operative to emit a signal responsive to the activation of the first switch, the electrode arranged on the bottom portion of the housing.

3. The device of claim 1, wherein the first finger is an index finger of the human hand.

4. The device of claim 1, wherein the first switch has a range of motion that is substantially linear along a line that intersects a portion of the electrode.

5. The device of claim 1, further comprising a mode selection switch that is operative to select a mode of operation of the device.

6. The device of claim 1, wherein a first mode of operation of the device includes outputting the signal at a first voltage and a second mode of operation of the device includes outputting the signal at a second voltage.

7. The device of claim 1, wherein the device includes an electronic portion that includes a first battery terminal and a second battery terminal, wherein a line defined by a point on the first battery terminal and a point on the second battery terminal is arranged substantially orthogonal to a plane defined the electrode and a biasing member.

8. The device of claim 1, wherein the device includes an electronic portion that includes a first battery terminal and a second battery terminal, wherein a line defined by a point on the first battery terminal and a point on the second battery terminal is arranged at an oblique angle to a plane defined by a point on the first switch and the electrode.

9. The device of claim 1, further comprising a textured surface region arranged between the electrode and a second electrode.

10. A transcutaneous electrical nerve stimulation device comprising:
a housing having a bottom portion and a top portion, the top portion including a curved palm-rest surface shaped to conform generally to the shape of a human palm of a human hand when the human hand is partially opened;
a first switch arranged on the top portion of the housing, the first switch being positioned to align in facing contact with a first finger of the human hand when the human hand is grasping the device, the first switch being operable by generally downward vertical movement of the first finger of the human hand; and
an electronic portion partially arranged in the housing, the electronic portion including:
a power source;
a boost converter communicatively connected to the power source;
a timer communicatively connected to the boost converter and a switching device;
a transformer communicatively connected to the boost converter and the timer; and
an electrode operative to emit a signal responsive to an activation of the first switch, the electrode arranged on the bottom portion of the housing.

11. The device of claim 10, wherein the first finger is an index finger of the human hand.

12. The device of claim 10, wherein the first switch has a range of motion that is substantially linear along a line that intersects a portion of the electrode.

13. The device of claim 10, further comprising a mode selection switch that is operative to select a mode of operation of the device.

14. The device of claim 10, wherein a first mode of operation of the device includes outputting the signal at a first voltage and a second mode of operation of the device includes outputting the signal at a second voltage.

15. The device of claim 10, wherein the device includes an electronic portion that includes a first battery terminal and a second battery terminal, wherein a line defined by a point on the first battery terminal and a point on the second battery terminal is arranged substantially orthogonal to a plane defined by the electrode and a biasing member.

16. The device of claim 10, wherein the device includes an electronic portion that includes a first battery terminal and a second battery terminal, wherein a line defined by a point on the first battery terminal and a point on the second battery terminal is arranged at an oblique angle to a plane defined by a point on the first switch and the electrode.

17. The device of claim 10, further comprising a textured surface region arranged between the electrode and a second electrode.

18. A transcutaneous electrical nerve stimulation device comprising:
a housing having a bottom portion and a top portion, the top portion including a curved palm-rest surface shaped to conform generally to the shape of a human palm of a human hand when the human hand is partially opened;
a first switch arranged on the top portion of the housing, the first switch being positioned to align in facing contact with an index finger of the human hand when the human hand is grasping the device, the first switch being operable by generally downward vertical movement of the index finger of the human hand; and
an electrode operative to emit a signal responsive to an activation of the first switch, the electrode arranged on the bottom portion of the housing.

19. The device of claim 18, wherein the first switch has a range of motion that is substantially linear along a line that intersects a portion of the electrode.

20. The device of claim 18, wherein the device includes an electronic portion that includes a first battery terminal and a second battery terminal, wherein a line defined by a point on the first battery terminal and a point on the second battery terminal is arranged substantially orthogonal to a plane defined by a point on the electrode and a biasing member.
